(19) United States
(12) Patent Application Publication
(10) Pub. No.: US 2009/0131907 A1
(43) Pub. Date: May 21, 2009

(54) Endoscopic Cardiac Surgery

(75) Inventors: Albert K. Chin, Palo Alto, CA (US); Patrick J. Massetti, San Mateo, CA (US)

Correspondence Address: FENWICK & WEST LLP, SILICON VALLEY CENTER, 801 CALIFORNIA STREET, MOUNTAIN VIEW, CA, 94041, US

(73) Assignee: MAQUET CARDIOVASCULAR LLC, San Jose, CA (US)

(21) Appl. No.: 12/347,802

(22) Filed: Dec. 31, 2008

Related U.S. Application Data
(62) Division of application No. 10/140,309, filed on May 6, 2002.
Application No. 10/140,309 is a continuation-in-part of application No. 09/635,721, filed on Aug. 9, 2000.
(60) Provisional application No. 60/150,737, filed on Aug. 25, 1999.
(60) Provisional application No. 60/148,130, filed on Aug. 10, 1999.

(51) Int. Cl. A61M 37/00; A61B 17/34; A61B 17/00

(52) U.S. Cl. 604/506; 604/176; 604/173; 606/185; 606/129

(57) ABSTRACT

Apparatus and surgical methods establish temporary suction attachment to a target site on the surface of a bodily organ for enhancing accurate placement of a surgical instrument maintained in alignment with the suction attachment. A suction port on the distal end of a supporting cannula provides suction attachment to facilitate accurate positioning of a needle for injection penetration of tissue at the target site on the moving surface of a beating heart. Force applied via the suction attachment to the surface of the heart promotes perpendicular orientation of the surface of the myocardium for enhanced accuracy of placement of a surgical instrument thereon. A hollow needle and a supporting channel therefor, each include a slot along an outer wall between ends thereof, are selectably rotatable to align the slots for releasing a cardiac lead from within the needle through the aligned slots.

### RELATED APPLICATION

This application is a divisional application of application Ser. No. 10/140,309, filed on May 6, 2002, which is a continuation-in-part of pending application Ser. No. 09/635,721, filed on Aug. 9, 2000 by A. Chin, which claims the benefit of the filing of provisional application Nos. 60/150,737, on Aug. 25, 1999, and 60/148,130 on Aug. 10, 1999, each of which applications is incorporated herein in its entirety by this reference.

### FIELD OF THE INVENTION

This invention relates to endoscopic cardiovascular surgical procedures and instruments, and more particularly to apparatus including a vacuum-assisted cannula and surgical instruments operable therewith, and to surgical procedures utilizing such apparatus.

### BACKGROUND OF THE INVENTION

The injection of undifferentiated satellite cells or myocytes or stem cells into the myocardium of a beating heart in the endoscopic procedure of cellular cardiomyoplasty must be performed carefully to avoid complications. A specialized instrument, as described in the aforecited applications, is advanced through an operating channel of an endoscopic cannula to deliver cells in controlled manner into a beating heart. If a needle is used to inject the cells, sufficient control must be provided to ensure that the needle does not puncture a coronary vein or artery and cause hemorrhage within the pericardial space, with subsequent cardiac tamponade. Movement of the beating heart further complicates needle placement because of erratic movement of the coronary vessels as needle insertion is attempted. Similarly, placement of other elements such as epicardial pacing or defibrillation leads into the myocardium of a beating heart must be carefully placed to avoid puncture of a coronary vein or artery with concomitant complications.

### SUMMARY OF THE INVENTION

In accordance with the illustrated embodiments of the present invention, a substantially rigid cannula includes separate elongated lumens extending between distal and proximal ends of the cannula to provide an instrument channel and one or more separate vacuum channels that terminate in a suction port located adjacent the distal end of the cannula. The instrument channel is sized to accommodate various surgical instruments including a hollow needle for penetrating the myocardium to deliver the cells. The needle is configured for shallow penetration to avoid puncturing into a chamber of the heart with associated complications. In an alternative embodiment, an instrument channel carried by a ‘needle’ is sized to accommodate epicardial pacing or defibrillating leads. Additionally, the cannula with separate lumens or channels therethrough may be in incorporated with or disposed within an instrument channel of an endoscopic cannula that houses an endoscope aligned with a distal transparent tip. This assemblage of surgical instruments may be conveniently positioned through tissue disposed between a subxiphoid incision and a surgical site on the epicardium of a beating heart, or positioned through tissue disposed between a thoracotomy incision and a surgical site on the epicardium of a beating heart. In some cases, a laterally expandable sheath may be employed to form a working cavity in tissue to facilitate the placement of the vacuum port and associated instrument channel at the surgical site on the epicardium.

### BRIEF DESCRIPTION OF THE DRAWINGS

FIG. 1 is a side view of a vacuum-assisted injection cannula in accordance with one embodiment of the present invention;

FIG. 2 is a side view of an endoscopic cannula for use with the injection cannula of FIG. 1;

FIG. 3 is a partial side view of the assembled cannulas of FIGS. 1 and 2 in a surgical procedure;

FIG. 4a is a partial side view of a split needle according to one embodiment of the present invention;

FIG. 4b is a partial side view of a needle with short bevel sharpened tip according to an embodiment of the present invention;

FIG. 5 is a perspective view of another embodiment of an injection cannula in accordance with the present invention;

FIGS. 6a and 6b comprise a flow chart illustrating a surgical procedure in accordance with the present invention;

FIG. 7 is a plan view of an epicardial lead with screw-like distal tip and attached proximal connector;

FIG. 8 is a partial plan view of a needle in one configuration incorporating an open instrument channel for placement of an epicardial lead;

FIG. 9 is a partial plan view of the needle of FIG. 8 in a complementary configuration incorporating a closed instrument channel; and

FIG. 10 is a plan view of a cannula with attached instrument channel.

### DETAILED DESCRIPTION OF THE INVENTION

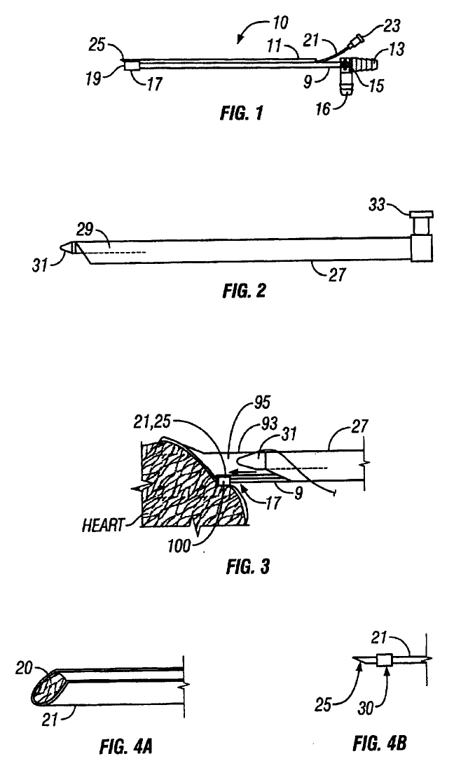

Referring now to FIG. 1, there is shown one embodiment of a suction assisted insertion cannula 10 according to the present invention including a closed channel 9 and a superior channel 11 attached to the closed channel. The closed channel 9 includes a suitable hose connection 13 and a three-way vacuum control valve 15 including an irrigation port 16 at the proximal end, and a suction pod 17 positioned on the distal end. The suction pod 17 includes a porous distal face or suction ports 19 that serves as a vacuum port which can be positioned against the epicardium to facilitate temporary fixation thereto as a result of the reduced air pressure of vacuum supplied to the suction pod 17. The distal end of the superior instrument channel 11 that is attached to the closed channel 9 may thus be held in accurate fixation in alignment with a selected surgical site on the epicardium relative to the suction fixation location of the suction pod 17 on the epicardium. A rounded smooth surface of suction pod 17 may be used to apply gentle pressure on the epicardium to stop bleeding at small puncture sites, or to allow injected cells to be absorbed without exiting back out of the injection.

The superior channel 11 is sized to accommodate slidable movement therein of a hollow needle 21 that may exhibit lateral flexibility over its length from the needle hub 23 at the proximal end to the sharpened distal end 25. When used to inject cells, the needle 21 may be about 22-25 gauge in diameter and includes an internal bore of sufficient size to facilitate injection of cells without incurring cell damage, or lysis. When used to place pacing or defibrillating leads, the needle 21 may be about 2-2.5 mm in diameter with an internal bore of sufficient size to accommodate a lead of diameter up to approximately 2 mm in diameter.

Due to the relatively large diameter of the needle for epicardial lead placement (approximately 2-2.5 mm in diameter), a solid obturator 20 may optionally be used with the slotted needle 21, as illustrated in FIG. 4a, for insertion into the myocardium. The obturator 20 closes off the distal end of the needle, to prevent the needle from coring out a section of the myocardium during needle insertion, with associated excessive bleeding. The obturator 20 may be removed from the needle 21 after needle insertion and the epicardial lead advanced into the myocardium. The epicardial lead, as illustrated in FIG. 7, is flexible and may be positioned within its own split sheath or tube for easier insertion through the slotted needle.

After the lead is implanted in the heart by the procedure described above, the proximal end is disposed out through the small initial incision in the patient. The proximal end may then be tunneled subcutaneously from the initial incision to an incision in the patient's upper chest where a pacemaker or defibrillator will be located. A small, elongated clamp is passed through the subcutaneous tunnel to grasp the proximal end of the epicardial lead to facilitate pulling the lead through the tunnel for placement and attachment to the pacemaker or defibrillator.

Both the superior channel 11 and the needle 21 may be longitudinally slotted for placing an epicardial lead that may incorporate a large diameter connector, as illustrated in FIG. 7. A split sheath can be used around the lead to facilitate advancement and rotation of the lead via the slotted needle. After anchoring such lead in the myocardium, for example by screwing in the distal tip, the slotted needle 21 is rotated to align its slot with the slot in the superior channel 11, thus allowing the lead to be released from the cannula.

The structure according to this embodiment of the invention, as illustrated in FIG. 1, is disposed to slide within the instrument channel in an endoscopic cannula 27, as shown in FIG. 2. This cannula includes an endoscope 29 therein that extends from a tapered transparent tip 31 attached to the distal end, to a viewing port 33 at the proximal end that can be adapted to accommodate a video camera. In this configuration, the structure as illustrated in FIG. 1 may be positioned within the instrument channel in the cannula 27 of FIG. 2 to position the suction pod 17 and sharpened needle tip 25 in alignment with a surgical target on the heart, as illustrated in FIG. 3. The suction pod 17 is temporarily affixed to the epicardium in response to suction applied to the porous face 19 of the suction pod 17 under control of a suction valve 15, and the sharpened tip 25 of the needle 21 may then be advanced to penetrate into the myocardium at an accurately-positioned surgical site, all within the visual field of the endoscope 29 through the transparent tip 31. Following injection, the needle is withdrawn and the suction pod 17 may be rotated or otherwise manipulated to position a surface thereof on the injection site with gentle pressure to allow time for the injected cells to be absorbed and to control any bleeding occurring out of the injection site.

As illustrated in FIGS. 2 and 3, the various channels in the endoscopic cannula 27 and the insertion cannula 10 have specific orientations with respect to each other in order to provide stabilization of the epicardial surface and allow visual control of the injection process. In the endoscopic cannula 27, the instrument channel is positioned below the endoscopic channel and this allows the cannula 27 and the transparent tapered tip 31 on the endoscope 29 to retract the pericardium away from the epicardial surface of the heart at the operative site. This creates a space 95 for contacting the heart below the pericardium, as illustrated in FIG. 3. As the cell insertion cannula 9 is advanced forward out of the instrument channel of the endoscopic cannula 27, the suction pod 17 is visualized through the endoscope 29 and transparent tip 31, as the suction pod 17 is placed on the epicardial surface of the heart. At a selected site on the heart, for example, at the site of an old myocardial infarct, the suction is activated to attach the pod 17 to the heart. The configuration of the instrument channel of the cell insertion cannula 10 on top of the suction channel 9 allows the needle 21 to be visible as soon as it exits from the instrument channel, and remain visible within the visual field of the endoscope along the entire path of travel of the needle 21 from the insertion cannula 10 to its insertion into the myocardium. Continuous visualization of the needle 21 in this manner helps to prevent inadvertent puncture of a coronary vessel.

The configuration of the suction pod 17 and the needle 21 on the insertion cannula 10 also facilitates delivery of substances or devices in an orientation perpendicular to the epicardial surface. For placement of pacing or defibrillation leads, it is particularly desirable to have the leads enter the myocardium in an orientation that is generally perpendicular to the epicardial surface for secure anchoring in the myocardium. Generally, the insertion cannula 10 is advanced through the endoscopic cannula 27 and approaches the epicardial surface of the heart at a tangential angle. Accordingly, the insertion cannula 10 is configured to facilitate deforming the epicardial surface in order to achieve perpendicular entry of the needle 21 into the myocardium, as illustrated in FIG. 3. The suction pod 17 of the insertion cannula 10 temporarily attaches to the epicardial surface upon application of vacuum under control of the valve 15. Downward pressure can be exerted on the epicardial surface via the substantially rigid insertion cannula 10. The pliable myocardium thus deforms to create a surface ledge 100 distal to the suction pod 17 oriented perpendicular to the axis of the superior instrument channel 11 of the insertion cannula 10, as illustrated in FIG. 3. As the needle 21 is advanced, it enters the myocardium generally perpendicularly to the epicardial surface as thus deformed for desirable lead placement or cell injection.

Referring now to FIGS. 3 and 4b, it should be noted that the insertion cannula 10 is sized to fit in slidable orientation within the working channel of about 5-7 mm diameter in the endoscopic cannula 27. The outer dimensions of the suction pod 17 are less than 5-7 mm diameter and is configured on the distal end of the closed channel 9 not to obstruct the forward movement of the needle 21 past the closed, back surface 19 of the suction pod 17.

As illustrated in FIG. 4b, the sharpened distal end 25 of the needle 21 includes a relatively short, sharpened bevel of length approximately 2-3 times the diameter of the needle. The short bevel length of the needle assures that cells are injected within the myocardium, and that part of the needle bevel does not extend into a heart chamber, with resultant intracardiac cell delivery. A visual and tactile marker 30 of extended diameter may be incorporated into the distal portion of the needle 21. As the needle is advanced into the myocardium, the marker 30 of enlarged diameter offers increased resistance to tissue insertion. The marker 30 is positioned just proximal to the bevel of the needle and extends proximally a distance of approximately 5-7 mm.

A needle stop may also be built into the proximal end of the needle 21. Such a stop may simply be the hub 23 of the needle, and the needle 21 may be sufficiently limited in length that only a specific length of needle, for example 1 cm, may extend out of the instrument channel of the cell insertion cannula 10 when the needle hub 23 abuts against the proximal face of the instrument channel 11. However, the distal visual and tactile marker 30 provides generally more precise guide to depth of needle penetration under conditions of different angles of possible needle insertion with respect to the epicardial surface. With an extremely shallow angle of entry, a needle of short length may not enter the heart at all. In use, the transparent tip 31 and the suction pod 17 of the assembled cell injection device may be manipulated to reshape a localized portion of the epicardial surface of the heart to allow perpendicular entry of the needle into the myocardium, as illustrated in FIG. 3. With the suction pod 17 activated, gentle manipulation of the insertion cannula allows adjustment of the needle entry angle while maintaining temporary vacuum-assisted attachment to the epicardial surface, as shown in FIG. 3.

The insertion device may also inject substances other than cells. Angiogenic agents such as vascular endothelial growth factor (VEGF) may be injected into myocardial scar tissue in an attempt to stimulate neovascularization, or growth of new blood vessels into the area. Insertion of the needle itself into myocardial tissue may be therapeutic as a form of transmyocardial revascularization (TMR). It is believed that needle insertion injury may stimulate angiogenesis, or growth of new vessels into a devascularized portion of the heart. The cell insertion cannula thus promotes accurate placement of a needle 21 into myocardium under continuous visualization. When combined with the endoscopic cannula, the needle placement may be accomplished through a small, 2 cm subxiphoid skin incision.

The illustrated embodiment of the insertion cannula includes a substantially rigid cannula containing a closed channel 9 ending in a distal suction pod 17, and a superior instrument channel 11 ending immediately proximal to the suction pod 17 on the closed channel 9. In operation, a long needle is advanced through the instrument channel 11. The needle 21 contains a marker 30 immediately proximal to its beveled tip 25 that serves as a visual or other sensory indicator of the depth of needle insertion. The marker 30 may be a segment of expanded diameter to provide tactile feedback upon insertion into myocardial tissue. For example, a gold-colored metallic sleeve 30 may be welded or soldered onto the needle 21 to provide both visual and tactile feedback of the depth of penetration of the needle tip into the myocardium. The marker may alternatively include a series of rings etched in the needle or a band etched or sandblasted in the same area. A three-way valve 15 on the cannula 9 allows suction in the pod 17 to be turned on or off, and allows irrigation fluid such as saline to be injected through the suction pod 17 while suction is turned off.

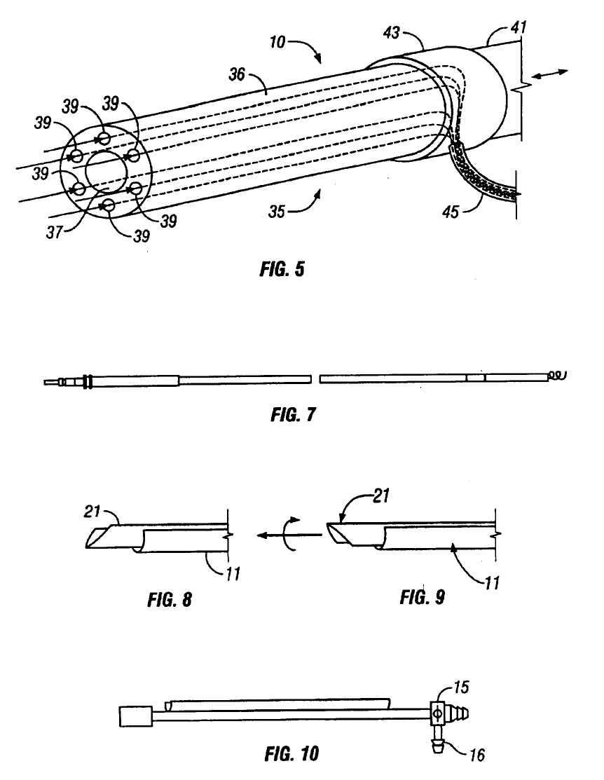

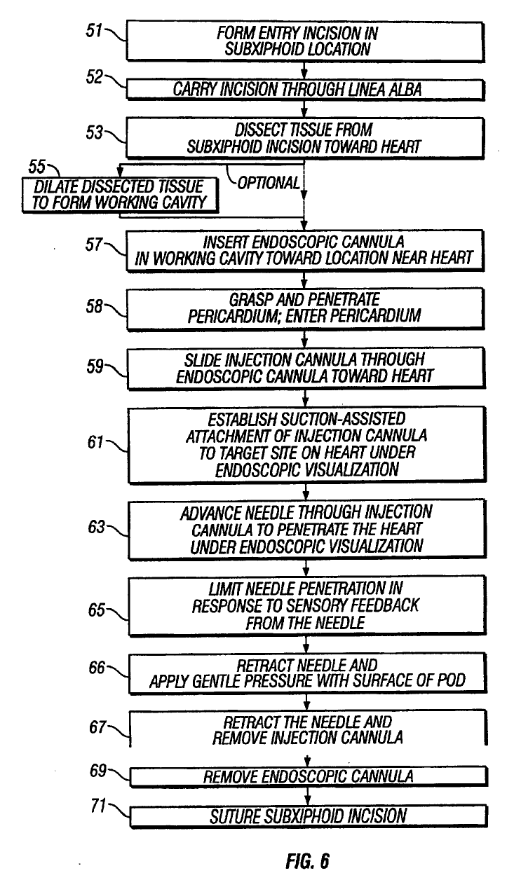

Referring now to FIG. 5, there is shown a perspective view of another embodiment of an insertion cannula 35 similar to insertion cannula 10 described above, including an elongated body 36 having a central bore 37 therethrough to serve as an instrument channel, and including one or more eccentric channels 39 that serve as suction conduits. The central bore may be sized to slidably support surgical instruments 41 therein such as tissue cutters and dissectors, electrocoagulators, injection needles, and the like. For example, surgical instrument 41 may be an energy-supplying ablation probe for epicardial ablation of myocardial tissue in the treatment of cardiac arrhythmia such as atrial flutter or atrial fibrillation. The ablation probe 41 may use radio frequency, microwave energy, optical laser energy, ultrasonic energy, or the like, to ablate myocardial tissue for arrhythmia correction. The suction pod 17 attaches to the epicardial surface while suction is turned on at valve 15 to facilitate advancing the ablation probe 41 through the cannula 35 into contact with the heart at the desired site under direct endoscopic visualization for precise myocardial ablation.

The left atrial appendage is frequently the site or source of thromboemboli (blood clots) that break away from the interior of the left atrial appendage and cause a stroke or other impairment of a patient. An ablation probe 41 can be used in the cannula 35 to shrink and close off the appendage to prevent thromboemboli from escaping.

In a similar procedure, a suture loop or clip can be placed through the cannula 35 and applied tightly around the atrial appendage to choke off the appendage.

The suction channels 39 in the cannula 35 of FIG. 5 may form a suction attachment surface at the distal end of the cannula 35, or may be disposed in fluid communication with a suitable suction pod with a porous distal face and with a central opening in alignment with the central bore 37. The suction-attaching distal face provides an opposite reaction force against a tool that exerts a pushing force such as a needle, screw-in lead tip, or other device deployed through the central bore 37 of the cannula 35. The proximal ends of the eccentric channels 39 are connected via a manifold or fluid-coupling collar 43 to a vacuum line 45. Alternatively, a single channel 39 may communicate with an annular recess or groove disposed concentrically about the central bore 37 within the distal end to serve as a suction-assisted attachment surface.

In this configuration, an injection needle 21 slidably disposed within the central bore 37 may be extended beyond the distal end of the cannula 35, within the visual field of an endoscope, in order to orient the needle in alignment with a surgical target site on the pericardium prior to positioning the distal end of the cannula on the pericardium and supplying suction thereto to temporarily affix the cannula 35 in such position. A cannula 35 formed of transparent bioinert material such as polycarbonate polymer facilitates visual alignment of the cannula 35 and the central bore 37 thereof with a surgical site, without requiring initial extension of a surgical instrument, such as a cell-injection needle, forward of the distal end within the visual field of an endoscope. In an alternative embodiment, the central lumen or bore 37 may serve as a suction lumen with multiple injection needles disposed in the outer lumens 39.

Referring now to the flow chart of FIGS. 6a, 6b, the surgical procedure for treating the beating heart of a patient in accordance with one embodiment of the present invention proceeds from forming 51 an initial incision at a subxiphoid location on the patient. The incision is extended 52 through the midline fibrous layer (linea alba). The tissue disposed between the location of subxiphoid incision and the heart is bluntly dissected 53, for example, using a blunt-tip dissector disposed within a split-sheath cannula of the type described in the aforecited patent application. The channel thus formed in dissected tissue may optionally be expanded 55 by dilating tissue surrounding the channel, for example, using a balloon dilator or the split-sheath cannula referenced above, in order to form a working cavity through the dissected and dilated tissue, although this may be unnecessary.

An endoscopic cannula, for example, as illustrated in FIG. 2 including an endoscope and a lumen for receiving surgical instruments therein is inserted 57 into the working cavity through the subxiphoid incision toward the heart to provide a field of vision around a target site on the heart, and to provide convenient access via the lumen for surgical instruments of types associated with surgical procedures on the heart. The first such instrument is the pericardial entry instrument, as described in the aforementioned provisional applications, which generally grasp the pericardium in a side-bite manner to form an elevated ridge of tissue through which a hole can be safely formed without contacting the epicardial surface. Once the pericardium is penetrated 58, other instruments can be inserted through the hole and into the working space 58. One such instrument is an insertion cannula, for example, as illustrated in FIG. 1, that includes a suction channel and an instrument channel and is slidably supported 59 within the instrument lumen of the endoscopic cannula. The suction channel of such instrument extends through the length thereof from a proximal end to a suction pod at the distal end that can be extended into contact 61 with the beating heart of the patient at a selected target site. The suction pod can be carefully positioned on the pericardium under visualization through the endoscope, and the suction can be applied to establish temporary attachment of the injection cannula to the pericardium. A needle or other surgical instrument such as surgical scissors or an electrocauterizer, or the like, is then moved into contact 63 with the pericardium to perform a surgical procedure at or near the target site. One surgical procedure includes penetrating the pericardium and myocardial tissue with the needle, typically in a region of a previous infarct, to stimulate transmyocardial revascularization or to inject undifferentiated satellite cells to promote regrowth of scarred myocardial tissue. During such surgical procedure, it is important to limit the depth of penetration of the needle in order to assure injection penetration only into the myocardium, and to avoid puncture into a heart chamber. A penetration indicator 30 may be disposed about the needle near the distal end thereof to provide visual and/or tactile feedback as mechanisms for limiting 65 the depth of needle penetration, as illustrated in FIG. 4b. Specifically, visualization of the penetration indicator via the endoscope facilitates control of manual extension of the needle into the myocardium. Additionally, an indicator of increased diameter disposed about the needle at an appropriate position proximal the distal end serves as a penetration indicator by providing increased tactile feedback of limiter by increasing the resistance to insertion of the needle into the myocardium. After needle penetration and cell injection, the suction pod 17 may be manipulated to apply gentle pressure 66 at a surface thereof to the injection site to allow cell absorption and to tamponade any bleeding from the injection site.

After one or more injections of the myocardium, positioned and performed as described above, the injection cannula and the needle supported therein are removed 67 through the instrument lumen of the endoscopic cannula which is then also retrieved 69 from the working cavity, and the initial subxiphoid entry incision is then sutured closed 71 to conclude the surgical procedure.

The endoscopic cannula and pericardial entry instrument may also be applied from a thoracotomy incision to gain access to the heart. A 2 cm incision is performed in an intercostal space in either the left or the right chest. Ideally, the incision is made between the midclavicular line and the anterior to mid axillary line. The incision is extended through the intercostal muscles and the pleura, until the pleural cavity is entered. The endoscopic cannula is then inserted into the pleural cavity and advanced to the desired area of entry on the contour of the heart, visualized within the pleural cavity. The pericardial entry instrument and procedure as described in the aforementioned applications are used to grasp the pleura, and a concentric tubular blade cuts a hole in the pleura, exposing the pericardium underneath. The pericardium is then grasped by the pericardial entry instrument, and the tubular blade is used to cut a hole in the pericardium, allowing access to the heart. The transparent tapered tip 31 of the endoscopic cannula 29 aids in pleural and pericardial entry by retracting lung and pleural tissue that may impede visualization of the pericardial entry site. Once the pericardium is entered, the endoscopic cannula 29 may be moved around to visualize anterior and posterior epicardial surfaces.

Therefore the surgical apparatus and methods of the present invention provide careful placement of an injection needle or other surgical instrument on the surface of a beating heart by temporarily affixing the distal end of a guiding cannula at a selected position on the heart in response to suction applied to a suction port at the distal end. The guiding cannula can be positioned through a working cavity formed in tissue between the heart and a subxiphoid or other entry incision to minimize trauma and greatly facilitate surgical treatment of a beating heart. Such treatments and procedures may include needle punctures of the myocardium, or injections therein of undifferentiated satellite cells, or other materials, to promote vascularization or tissue reconstruction, for example, at the site of a previous infarct. Such treatments and procedures may also include placing of pacing or defibrillating leads into the myocardium. Such treatments and procedures may further include positioning and manipulation of an ablation probe to ablate myocardial tissue and correct cardiac arrhythmias.

What is claimed is:

1. A method of performing a surgical procedure on the heart of a patient under visualization through an endoscope, the method comprising:
establishing a working cavity through tissue between the heart and an entry location;
inserting through the entry location and in the working cavity a first cannula including an instrument channel disposed between proximal and distal ends thereof and including an endoscope positioned in the first cannula to provide a visual field forward of the distal end;
slidably positioning a second cannula in the instrument channel of the first cannula, with a channel of the second cannula extending between distal and proximal ends thereof, and with a suction port positioned on the distal end of the second cannula;
contacting a target site on the heart with the suction port, and supplying suction thereto; and
extending an instrument through the channel of the second cannula beyond the distal end of the second cannula into contact with the heart within the visual field of the endoscope.

2. The method according to claim 1 in which the entry location is a subxiphoid location.

3. The method according to claim 1 in which a thoracotomy is performed at the entry location.

4. The method according to claim 1 in which extending an instrument includes passing a needle through the channel of the second cannula and extending a distal end of the needle to penetrate the heart to a selected depth.

5. The method according to claim 4 in which the needle includes a bore therethrough and includes a sharpened distal end for penetrating the heart to the selected depth to inject a substance therein.

6. The method according to claim 5 in which the needle penetrates the myocardium of the heart to inject therein undifferentiated satellite cells, myocytes, or stem cells.

7. The method according to claim 4 in which the needle penetrates the myocardium of the heart to place therein a conductive lead for electrical pacing or defibrillation of the heart.

8. The method according to claim 7 in which the channel in the second cannula and needle each includes an elongated slot extending between distal and proximal ends thereof, and including after placing the conductive lead, rotating the needle to align the elongated slot therein with the elongated slot in the channel of the second cannula for releasing the conductive lead retained therein through the aligned slots.

9. The method according to claim 4 in which the channel of the second cannula is disposed eccentric the suction port within the visual field of the endoscope.

10. A method of performing a surgical procedure on the heart of a patient under visualization through an endoscope, the method comprising:
forming a working cavity in tissue between the heart and a subxiphoid entry location;
advancing a surgical instrument including the endoscope through the subxiphoid entry location and working cavity toward the heart;
establishing a suction attachment to a target site on the epicardium beneath the pericardium under visualization through the endoscope; and
contacting the epicardium beneath the pericardium at a location referenced to the target site of the suction attachment for performing a surgical procedure thereat under visualization through the endoscope.

11. The method according to claim 10 in which contacting the heart includes penetrating myocardial tissue of the heart at the referenced location.

12. The method according to claim 11 in which penetrating myocardial tissue at the referenced location includes inserting a needle to selected depth.

13. The method according to claim 12 including injecting material through the needle into myocardial tissue.

14. The method according to claim 13 in which the injected material includes undifferentiated satellite cells or myocytes or stem cells; and
the referenced location includes a site of previous infarct in the myocardium.

15. The method according to claim 12 including placement of a conductive lead into the penetrated myocardial tissue for electrical pacing or defibrillation of the heart.

16. The method according to claim 15 in which the conductive lead is confined within the needle having an elongated slot along an outer wall thereof between proximal and distal ends thereof, and including a support for the needle having an elongated slot along an outer wall thereof between proximal and distal ends thereof, the method further comprising:
rotating the needle after placement of the conductive lead to align the slots in the needle and support for releasing the conductive lead through the aligned slots.

17. The method according to claim 10 in which contacting the heart includes applying an ablation probe to the epicardial surface.

18. The method according to claim 12 in which the needle includes a penetration indicator for providing sensory indication of depth of penetration.

19. The method according to claim 18 in which the penetration indicator provides indication visible through the endoscope of the depth of needle penetration.

20. The method according to claim 18 in which the penetration indicator includes a segment of the needle of extended dimension at a location thereon that is proximal a distal end for providing tactile feedback indicative of the depth of penetration to said segment.

21. The method according to claim 10 in which the referenced location is laterally displaced toward the endoscope from the target site of the suction attachment.

22. The method according to claim 10 in which the referenced location is substantially concentrically disposed within the target site of suction attachment.

23. The method according to claim 10 including applying downward force at the site on the epicardial surface at which suction attachment is established for deforming myocardium thereat substantially perpendicular to the orientation of contact therewith.

24. The method according to claim 10 in which the target site of suction attachment is laterally displaced from, and within the visual field of the endoscope.

25. The method according to claim 10 in which the working cavity is formed by dissecting tissue from the subxiphoid entry location along a path toward the heart, and then by dilating the dissected tissue to form the working cavity.

26. Surgical apparatus comprising:
an elongated cannula having first and second separate channels therein and including a suction port at a distal end of the elongated cannula in fluid communication with the first lumen; and
the second lumen having a distal end thereof displaced from the suction port for slidably extending a surgical instrument therethrough forward of the suction port.

27. Surgical apparatus as in claim 20 in which the second lumen is disposed eccentric the first lumen and is dimensioned for slidably supporting a needle therein to selectively extend a distal end of the needle forward of the suction port.

28. Surgical apparatus according to claim 20 in which the second channel includes an elongated slot therein between distal and proximal ends thereof, and dimensioned for slidably and rotatably supporting therein a needle including an elongated slot therein between distal and proximal ends thereof to selectively extend the distal end of the needle forward of the suction port.

29. Surgical apparatus as in claim 26 in which the second lumen is substantially concentrically disposed within the first lumen for slidably supporting a surgical instrument in the second lumen to extend forward of the suction port.

30. Surgical apparatus as in claim 29 in which the suction port includes an annulus area at the distal end of the elongated cannula surrounding the second lumen to form a contact surface for suction attachment thereof to a surface of a bodily organ.

31. Surgical apparatus as in claim 26 in which the surgical instrument comprises a needle dimensioned to slide within the second lumen and includes a distal end skewed from perpendicularity to form a sharpened substantially planar end surface having a length not greater than about 3 times the diameter dimension of the needle.

32. Surgical apparatus as in claim 26 in which the surgical instrument includes a needle dimensioned to slide within the second lumen and to penetrate the myocardium of the heart, and including a penetration indicator disposed relative to the distal end of the needle to provide indication of depth of penetration of the myocardium.

33. Surgical apparatus as in claim 32 in which the penetration indicator includes a band disposed about the needle at a location proximal the distal end of the needle to provide visual indication of depth of penetration into the myocardium.

34. Surgical apparatus as in claim 32 in which the penetration indicator includes a segment of the needle having extended diametric dimension to provide tactile indication of increased resistance to penetration of the myocardium at a depth of penetration related to the location of the segment with respect to the distal end of the needle.
